(19) United States
(12) Patent Application Publication
(10) Pub. No.: US 2014/0010794 A1
(43) Pub. Date: Jan. 9, 2014

(54) Formulation and Method for Preparing Specific T Cell, and Method for Preparing the Formulation

(71) Applicant: FullHope Biomedical Co., Ltd., Taipei City (TW)

(72) Inventor: Jan-Mou LEE, Taipei City (TW)

(21) Appl. No.: 13/918,762

(22) Filed: Jun. 14, 2013

(30) Foreign Application Priority Data
Jul. 5, 2012 (TW) 101124293

(51) Int. Cl. C12N 5/0783

(52) U.S. Cl. CPC C12N 5/0636
USPC 424/93.71; 435/325; 435/372; 435/375

(57) ABSTRACT

The present invention provides a formulation and method for preparing specific T cells, and a method for fabricating the above formulation is also disclosed. The formulation can induce specific T cell responses and comprises at least a cell population of dendritic killer cells presenting specific antigens. In addition, the method mentioned above for preparing specific T cells comprises following steps. A cell population of T cells is provided at first. And then, a formulation of preparing specific T cells is added to mix with the cell population of T cells. After cultivating, the specific T cells are harvested. Furthermore, the method for fabricating the above formulation comprises the following steps. First, a cell population of dendritic killer cells is provided. A target sample is then provided, and a step of making the cell population of the dendritic killer cells co-cultivate with the target sample is performed. After co-cultivating, a cell population of dendritic killer cells presenting specific antigens is harvested.

### CROSS-REFERENCE TO RELATED APPLICATIONS

This Non-provisional application claims priority under 35 U.S.C. §119(a) on Patent Application No(s). 101124293 filed in Taiwan, Republic of China, Jul. 5, 2012, the entire contents of which are hereby incorporated by reference.

### FIELD OF THE INVENTION

This invention relates to a formulation/method for preparing specific T cells and a method for preparing the above formulation, especially relates to a formulation/method for preparing specific T cells and a method for preparing the above formulation by utilizing dendritic killer cells which can present specific antigens to induce T cells response.

### BACKGROUND OF THE INVENTION

Human body will recognize the extraneous matter and start a series of defending process. This defense system is named as immune system. There are many different cells such as leukocytes and lymphocyte, and different protein factors such as immunoglobulins and cytokines working coordinately to protect the body. The immune systems are traditionally divided into innate and adaptive immune systems. Innate immune system is including soluble complement system, polymorphonuclear neutrophils, macrophages and natural killer cells. Adaptive immune system is including humoral and cellular immunity. Humoral immunity as well as cellular immunity involves lymphocyte, lymphokine and immunological memory system. The long-lasting immune memory mounts quick and strong immune responses towards the same pathogen which has invaded the body.

Immune system may respond to different pathogens due to the diversity of major histocompatibility complex (MHC) molecules. The endogenous and exogenous antigens derived from pathogens, are assembled with MHC molecules on the surface of antigen-presenting cells (APC) and then presented to T cells expressing corresponding T cell receptors. MHC in the human beings can be called Human Leukocyte Antigen, HLA, which can be categorized into class I, class II, and class III. HLA class I is widely expressed on all the somatic cells but Class II distribution is restricted to macrophages, B cells and dendritic cells.

Dendritic cells (DC), which have the broadest range of antigen presentation, are professional APC, and named by the appearance of dendrites extending from the cell body. DCs reside in the periphery of body as immature DCs (imDCs). Once pathogen invades human bodies, imDCs capture pathogen-derived antigens, migrate to draining lymph nodes to become mature DCs (mDCs), and present antigens to corresponding T cells there. Therefore, dendritic cells are the starter of the pathogen-specific cellular immune responses.

Natural killer (NK) cells, a key player of innate immune system, spontaneously kill tumor or virally infected cells prior to activation. Mechanisms underlying cytotoxicity of NK cells are grouped into two parts: a) interaction of cell surface tumor necrosis factor superfamily members and their receptors which leads to apoptosis of target cells, (b) release of soluble perforin and granzymes. NK cells are rich with small granules in their cytoplasm contain special proteins such as perforin and proteases known as granzymes. Upon release in close proximity to a cell slated for killing, perforin forms pores in the cell membrane of the target cell through which the granzymes and associated molecules can diffuse in, leading to destruction of target cells. Once virally infected cells or tumor cells have been killed, viral genomic content (CpG or poly I:C), cellular metabolites, and bystander cytokines such as IFN-γIL-12 and TNF-α would further activate and augment NK cell activity in term of cytotoxicity and effector cytokine production. Therefore NK cells serve as key innate effector cells targeting to virally infected cells and tumor cells in a non-antigen specific manner while DCs in adaptive immune system trigger antigen-specific cytotoxic T cells which can further clear the infection. Patients deficient in NK cells are proved to be highly susceptible to early phases of herpes virus infection.

Interferon-producing killer dendritic cells (IKDCs), a recently identified leukocyte population in mice, express phenotypes of non-T (CD3⁻), non-B (CD19⁻), intermediate levels of CD11c, and high levels of B220 and NK-specific markers, including NK1.1, DX5, NKG2D and Ly49 family receptors. IKDCs functionally resemble NK cells in cytotoxicity against tumor cells and in production of abundant IFN-γ. On the other hand, upon stimulation with CpG or tumor cells. IKDCs down-regulate NKG2D, up-regulate MHC II, and acquire moderate APC-like activity that activates antigen-specific T cells. Despite acquisition of APC activity after certain stimulations, IKDCs appear to belong to the NK lineage rather than DC lineage. IKDCs express NK-specific Ncr-1 transcripts (encoding NKp46) but not PU.1 that is predominantly expressed in DCs and plasmacytoid DCs. Furthermore, IKDC development parallels NK cells in their strict dependence on the IL-15 cytokine system. Therefore, the putative IKDCs are functionally and developmentally similar to NK cells. Although debates regarding tumoricidal activity and cell lineage development of IKDC were raised herein, further investigations were limited by rare abundance of IKDC in periphery. The frequency of IKDCs in a mouse spleen is below 0.01%, and is even lower in the lymph nodes. Therefore, cumbersome procedure is required for the purification of IKDCs, and the yield is low. This problem has limited the use of IKDCs in research and in application.

Thus, Applicant put a lot of efforts in the past years and successfully screens out cells which have the functions of both natural killer cells and dendritic Cells. The abovementioned cells are defined as Dendritic Killer Cell (hereafter called DKC), also be called cytotoxic dendritic cell (cytoDC).

However, it is noted that the DKC constitutes less than 0.01% of peripheral lymphocytes. Please refer to FIG. 1 to FIG. 2B; FIG. 1 is a diagram showing the percentage of DKC in human peripheral blood of the cancer patient and the healthy donor FIG. 2A is a diagram showing the result of using a flow cytometer to analyze human peripheral blood of a cancer patient in the preferred embodiment of the present invention, and FIG. 2B is a diagram showing the result of using a flow cytometer to analyze human peripheral blood of a healthy donor in the preferred embodiment of the present invention. As shown in FIG. 1, the percentage of the DKC in human peripheral blood of the cancer patient is obviously lower than that of the healthy donor. Furthermore, as shown in the upper right corner of FIG. 2A, it is noted that the percentage of the DKC 10 in the human peripheral blood of the cancer patient is only 0.0367%. However, as shown in the upper right corner of FIG. 2B, the percentage of the DKC 10 in the human peripheral blood of the healthy donor is 0.436%. That is, the counts of the DKC which are existed in the human peripheral blood of the healthy donor are higher than that of the cancer patient.

On the other hand, tumors will change their antibodies to avoid identifying from immune cells so that the curative effect of the immune cell therapy cannot reach an original expectation.

### SUMMARY OF THE INVENTION

According to the abovementioned disadvantages of the prior art, Applicant successfully makes trace DKC of human blood increase in an amount of 200-fold to 400-fold, and further make the expanded DKC acquire antigen-presenting cell activities. A formulation will be prepared by combing the abovementioned DKC presenting specific antigens and a physiologically acceptable medium or buffer, and activates T cells to form specific T cells.

The present invention provides a formulation for preparing specific T cells, and the formulation comprises at least a cell population of dendritic killer cells presenting specific antigens.

Preferably, the formulation further comprises a physiologically acceptable medium or buffer. Preferably, the cell population of dendritic killer cells has a concentration of 10⁶ cell/mL. Preferably, the specific antigens are tumor-specific antigens.

The present invention further provides a composition containing a plurality of tumor antigen-pulsed human DKCs, wherein the plurality of tumor antigen-pulsed human DKCs is obtained by contacting a population of human DKCs with cells derived from a tumor, the population of human DKCs being HLA-G⁻ CD14⁻ CD19⁻ CD3⁻ CD56⁺ HLA-DR⁺.

The present invention further provides a method for preparing the abovementioned formulation, and the method comprises the following steps. First, a cell population of dendritic killer cells is provided. A target sample is then provided, and a step of making the cell population of dendritic killer cells co-cultivate with the target sample is performed. After co-cultivating, a cell population of dendritic killer cells presenting specific antigens is harvested.

Preferably, the cell population of dendritic killer cells processes a cytotoxic reaction against the target sample and presents the specific antigens of the target sample on the surface. The DKCs became antigen-loaded antigen-presenting cells.

Preferably, the target sample is tumor cells from a cancer patient, and the specific antigens are tumor-specific antigens. Preferably, the target sample and the cell population of dendritic killer cells are both gathered from the cancer patient.

Preferably, the cell population of the dendritic killer cell is generated ex vivo by culturing dendritic killer cells from a human blood sample with a cytokine, and the cytokine comprises IL-15.

Preferably, the method is a platform for screening specific antigens.

The present invention further provides a method for preparing specific T cells, and the method comprises the following steps. A cell population of T cells is provided at first. And then, a formulation of preparing specific T cells is added to mix with the cell population of T cells. After cultivating, the specific T cells are harvested.

Preferably, the formulation comprises a cell population of dendritic killer cells presenting specific antigens.

Preferably, the cell population of T cells is activated by the cell population of dendritic killer cells presenting specific antigens to form the specific T cells, and the specific antigens are tumor-specific antigens. Preferably, the specific T cells are tumor-specific T cells.

The present invention further provides a method of treating a tumor in a human subject in need thereof, the method comprising: First, provide a plurality of tumor antigen-pulsed human DKCs that is prepared by contacting a population of human DKCs with cells derived from a tumor in the subject, the population of DKCs being HLA-G⁻ CD14⁻ CD19⁻ CD3⁻ CD56⁺ HLA-DR⁺. Then, contact a population of CD8⁺ T-cells with the plurality of tumor antigen-pulsed human DKCs to generate tumor antigen-specific CD8⁺ T-cells. Finally, deliver the tumor antigen-specific CD8⁺ T-cells to the human subject.

The features and advantages of the present invention will be understood and illustrated in the following specification and FIGS. 1˜10B.

### BRIEF DESCRIPTION OF THE DRAWINGS

FIG. 1 is a diagram showing the percentage of DKC in human peripheral blood of the cancer patient and the healthy donor;

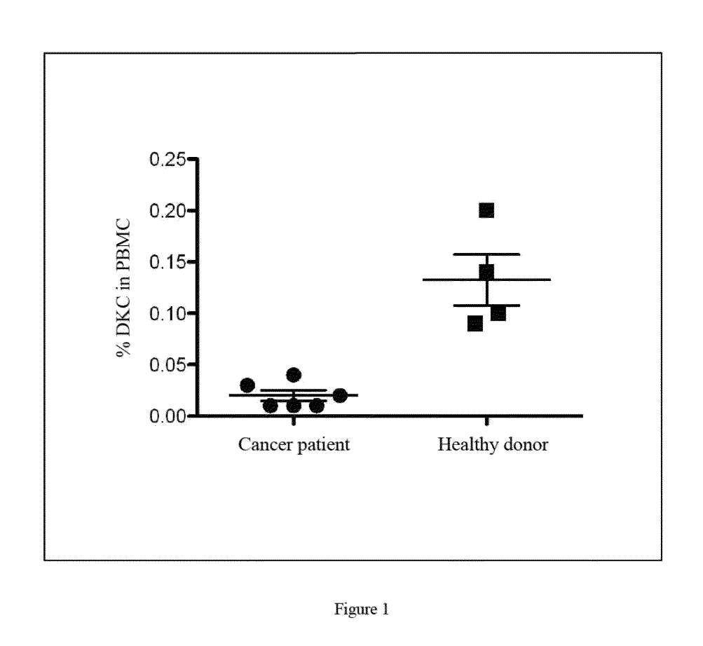

FIG. 2A is a diagram showing the result of using a flow cytometer to analyze human peripheral blood of a cancer patient in the preferred embodiment of the present invention;

FIG. 2B is a diagram showing the result of using a flow cytometer to analyze human peripheral blood of a healthy donor in the preferred embodiment of the present invention;

FIG. 3 is a flow chart showing a method according to an embodiment of the present invention for cultivating DKC population;

FIG. 4A to FIG. 4C are diagrams showing the results of detecting and screening cultivated DKC population by a flow cytometer;

FIG. 5 is a diagram showing an operational mechanism of DKC;

FIG. 6 is a flow chart showing a method according to an embodiment of the present invention for fabricating a formulation of preparing specific T cells;

FIG. 7A to FIG. 7B are diagrams showing different conditions of ovarian cancer cells before and after adding DKC population therein;

FIG. 8 is a flow chart showing a method according to an embodiment of the present invention for preparing specific T cells;

FIG. 9A and FIG. 9B are diagrams showing the result of activating T cells by the cell population of the dendritic killer cells presenting specific antigens according to an embodiment of the present invention; and

FIG. 10A and FIG. 10B are diagrams showing the result of activating T cells by the cell population of the dendritic killer cells without reacting with the tumor cells.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

As used herein, the term “Dendritic killer cells” or “DKC” is intended to refer to the cells with both cytotoxicity and antigen presenting cell (APC) activity.

As used herein, the term “Dendritic killer cells presenting specific antigens” means that the surfaces of the dendritic killer cells have presented the specific antigens.

As used herein, the term “antigen-pulsed human DKC” means that the human DKC have presented the specific antigen on its surface.

As used herein, the term “specific T cells” or “antigen-specific CD8⁺ T-cell” means that T cells activated by antigen-presenting cells are specific for the antigen presented by the above antigen-presenting cells.

As used herein, the symbol “+” means that the cell surface marker expresses on the surfaces of the cells and has a larger expressed amount measured by flow cytometer than that of the negative control.

As used herein, the symbol “−” means that the cell surface marker does not express on the surfaces of the cells and has an expressed amount equal to that of the negative control.

Preferably, all abovementioned expressed amount of the cell surface markers are measured by flow cytometer; however, the present invention is not limited thereto.

As used herein, the term “Interleukin” means a group of cytokines that were first seen to be expressed by white blood cells (leukocytes). It has since been found that interleukins are produced by a wide variety of body cells. The function of the immune system depends in a large part on interleukins.

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention. To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Preferably, several sorting or screening steps are performed by a flow cytometer, and a target cell population will be screened out by utilizing at least one flow cytometer to identity different surface markers of different cells. Flow cytometry allows for single cell analysis at speeds far surpassing any other single cell analysis technology in the art. This enables a statistically significant number of cells to be analyzed faster than using other alternative techniques. In a preferred embodiment, a flow cytometer is used with any suitable sample preparation robot or liquid handler that is known in the art. Furthermore, a single laser flow cytometer is used in an embodiment for the analyzing step. In another embodiment, a multi-laser flow cytometer is used for the analyzing step and the present invention is not limited thereto.

At first Applicant put a lot of efforts in the past years and successfully screens out cells which have the functions of both natural killer cells and dendritic Cells. These cells are defined as dendritic killer cell (hereafter called DKC) as mentioned above and have surface markers of HLA-G⁻CD14⁻CD19⁻CD3⁻CD56⁺ HLA-DR⁺.

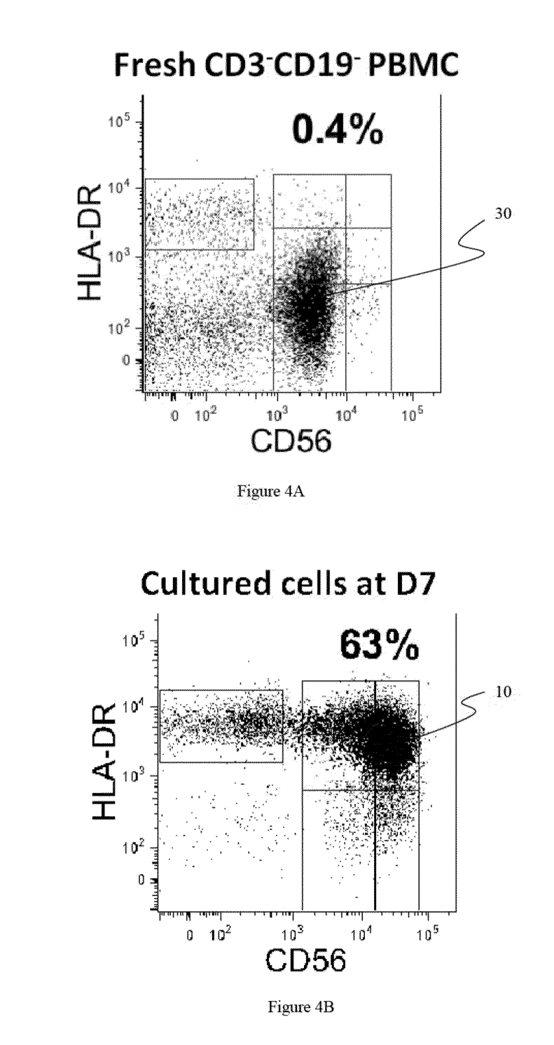

As abovementioned, the DKCs are identified from human peripheral blood. Please refer to FIG. 3; FIG. 3 is a flow chart showing a method for cultivating DKC population. First, step S100 of obtaining a peripheral blood mononuclear cell population from human blood is performed. And then, step S101 of adding an effective amount of at least a cytokine to mix with the peripheral blood mononuclear cell population is performed. Preferably, the cytokine comprises an effective amount of Interleukin-15 (hereafter “IL-15”). The following step S102 is to place the peripheral blood mononuclear cell population for an appropriate period. Finally, a DKC population will be sorted in step S103.

Preferably, the abovementioned cytokine further comprises Interleukin-12 (hereafter “IL-12”). Preferably, the concentration of abovementioned IL-15 is 10 ng/mL, and the concentration of IL-12 is a value between 0.5˜20 ng/mL.

Preferably, the abovementioned step S100 further comprises the following steps. At first, the 40 peripheral blood is gathered and the human peripheral blood mononuclear cell (hereafter “PBMC”) is sorted. T cells and B cells are then removed from the peripheral blood mononuclear cell population. Finally, the concentration of the peripheral blood mononuclear cell population will be adjusted to 10⁶ cell/mL. The human peripheral blood mononuclear cells comprise the following five categories of cells: monocytic cells, small cells, lymphoid cells, large cells and large and granular cells. Flow cytometry can be first used to select one or more types of cells for follow steps. The cell preferably comprises monocytic cells or lymphoid cells or both, and the monocytic cell population will be used as a preferred embodiment in the following. However, the present invention is not limited thereto.

Preferably, the abovementioned appropriate period means that IL-15 and the peripheral blood mononuclear cell population are both put into a media for a period to let cell proliferation process. Preferably, the appropriate period is the seventh day after starting the abovementioned cultivating step.

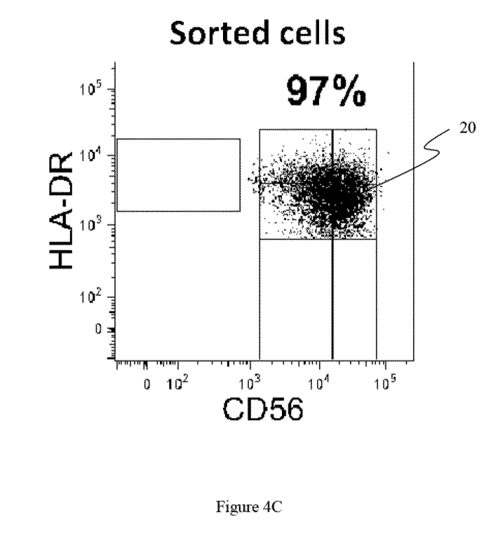

Please refer to FIG. 4A to FIG. 4C; FIG. 4A to FIG. 4C are diagrams showing the results of detecting and screening cultivated DKC population by a flow cytometer. First, FIG. 4A illustrates the results of detecting the surface markers of CD56 and HLA-DR by a flow cytometer after removing the T cell and B cell (CD3⁻CD19⁻PBMC) from human peripheral blood mononuclear cells and before cultivating. FIG. 4B illustrates the results of detecting the surface markers of CD56 and HLA-DR by a flow cytometer at the seventh day after starting the cultivating step. FIG. 4C illustrates the results of sorting DKC population by a flow cytometer. After removing T cells and B cells from the mononuclear cells of human blood, 10 ng/mL IL-15 and 2 ng/mL IL-12 are added. The cultivated DKC is harvested after seven days.

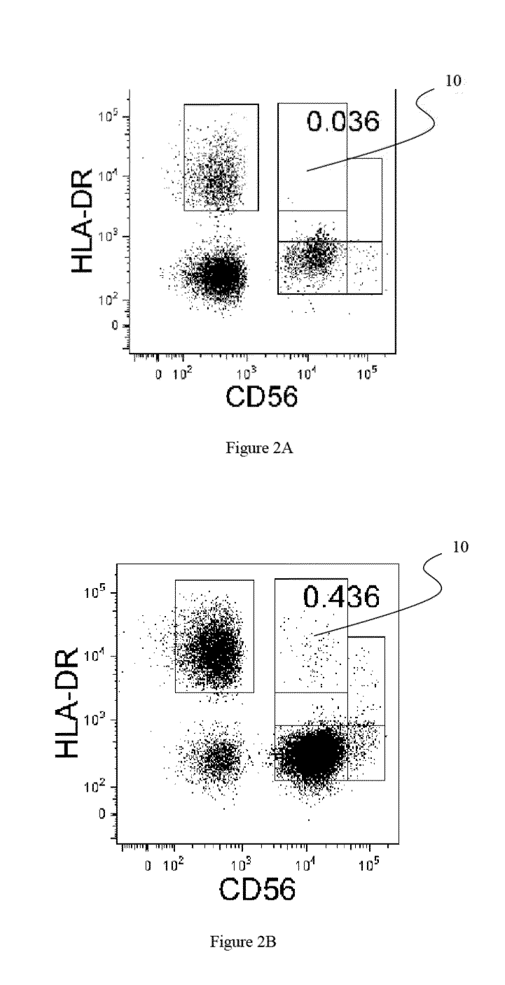

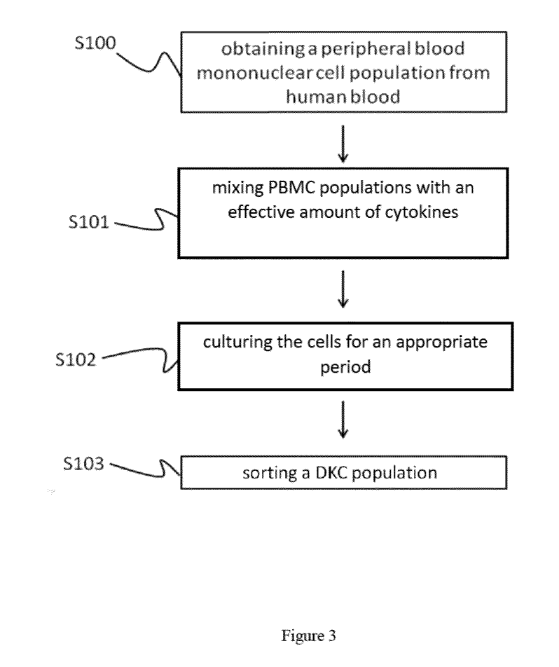

As shown in FIG. 4A, the counts of the cells which have natural killer cell surface marker (CD56⁺) and dendritic Cell surface marker (HLA-DR⁺) are much fewer. And further, the cells 30 positioned at the central portion are natural killer cells which have the surface marker of CD 56 but not HLA-DR. Please refer to FIG. 2B, the cells will transfer to the DKC 10 which has both natural killer cell surface marker (CD56⁺) and dendritic cell surface marker (HLA-DR⁺). The DKCs expand the counts and further let natural killer cells transfer to DKC. Finally, FIG. 4C illustrates the sorted cells selected by flow cytometer, the DKC population 20 which has the surface marker of HLA-G⁻CD14⁻CD 19⁻CD3⁻CD56⁺HLA-DR⁺.

It is noted that the abovementioned appropriate period is the preferred embodiment; however, the present invention is not limited thereto. That is, the step S103 can be performed on the fourth day after cultivating or on the tenth day after cultivating. Furthermore, the steps S101˜S103 can be repeatedly performed after the step S103. That is, non-adherent cells will be collected again and the counts of the dendritic killer cells will be expanded to an expect value by repeating the abovementioned steps.

Preferably, the method disclosed in the present invention is processed ex vivo, wherein the human blood is collected from a cancer patient. And further, a cancer, which the cancer patient suffers from, can be selected from a group consisting of squamous cell carcinoma, lobular carcinoma in situ, liver cancer, nasopharyngeal carcinoma, lung cancer, bone cancer, pancreatic cancer, skin cancer, head and neck cancers, malignant melanoma, cervical cancer, ovarian cancer, colon cancer, anal cancer, stomach cancer, breast cancer, testicular cancer, fallopian tube cancer, endometrial cancer, cervical cancer, vaginal cancer, vulvar cancer, non-Hodgkin lymphoma, Hodgkin lymphoma, esophageal cancer, thyroid cancer, adrenal cancer, cancers of mesothelial and soft tissue, urethra cancer, cancer of penis, prostate cancer, acute leukemia, chronic leukemia, lymphomas, bladder cancer, ureteral cancer, renal cell carcinoma, urothelial carcinoma, cancer of central nervous system, primary central nervous system lymphoma, glioma, pituitary tumor, Kaposi's sarcoma, squamous cell cancer and their metastasis.

In the following, the present invention provides a formulation for preparing specific T cells, and the formulation comprises at least a cell population of dendritic killer cells presenting specific antigens. Furthermore, the formulation comprises a physiologically acceptable medium or a physiologically acceptable buffer. It is noted that the cell population of the dendritic killer cells presenting specific antigens is obtained by further dealing with the abovementioned cultivated DKC population, and the cell population of the dendritic killer cells has absolutely different characteristics from the DKC population.

It is needed to be further illustrated that the cell population of the dendritic killer cells included within the formulation is a cell population of dendritic killer cells presenting specific antigens. And further, the formulation is used to induce specific T cell responses. That is to say, the present invention also discloses a method for preparing specific T cells by utilizing the abovementioned cell population of the dendritic killer cells presenting specific antigens. The above formulation and method for preparing specific T cells, and method for preparing the formulation are both illustrated with figures as the following.

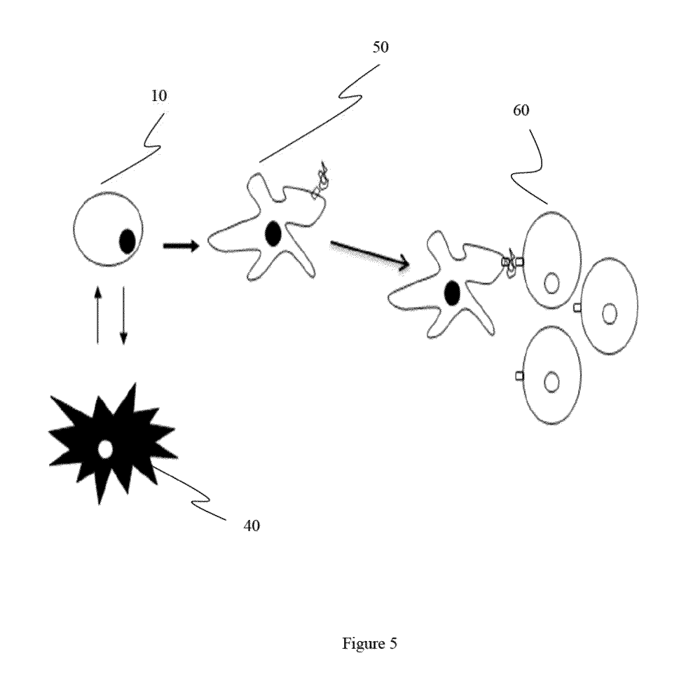

Please refer to FIG. 5; FIG. 5 is a diagram showing an operational mechanism of DKC. As shown in the figure, the dendritic killer cells 10 can identify tumor cells 40. After identifying, the dendritic killer cells 10 will produce interferon-γ (hereafter “IFN-γ”) to kill the tumor cells 40 by their cytotoxicity and further swallow the fragments of the killed tumor cells 40 to present tumor-specific antigens on their surface for forming the cell population of the dendritic killer cells presenting specific antigens 50. The above cell population of the dendritic killer cells presenting specific antigens 50 will present the tumor-specific antigens to CD8⁺ T cells 60 to activate CD8⁺ T cells. After activating, the CD8⁺ T cells become specific T cells.

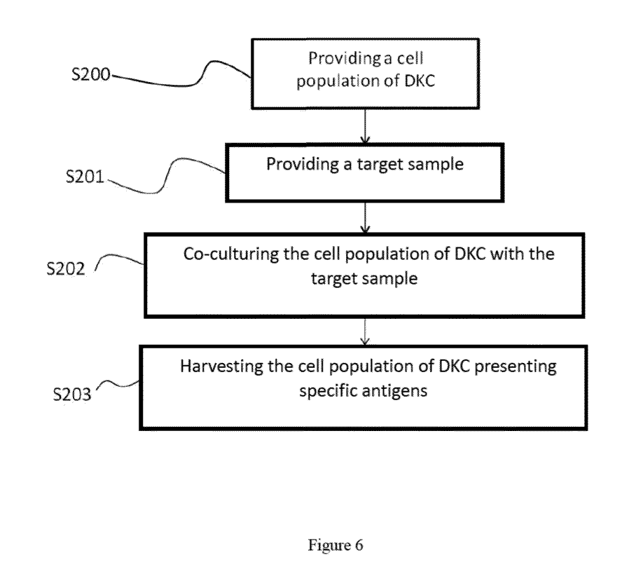

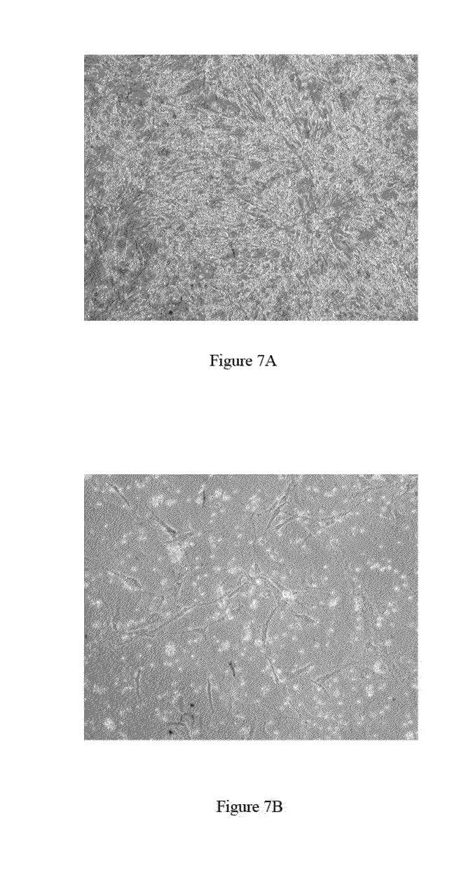

Please refer to FIG. 6, FIG. 7A and FIG. 7B. FIG. 6 is a flow chart showing a method according to an embodiment of the present invention for preparing a formulation of preparing specific T cells, and FIG. 7A to FIG. 7B are diagrams showing different conditions of ovarian cancer cells before and after adding DKC population therein.

As shown in FIG. 6, the cultivated cell population of the dendritic killer cells is obtained in the step S103, and the cultivated cell population of the dendritic killer cells can be further used in the following method for preparing the above formulation in step S200. A target sample is then provided in step S201. Preferably, the target sample is the tumor cell gathered from the same cancer patient. For example, the target sample is ovarian cancer cells of the abovementioned cancer patient and the ovarian cancer cells obtained from patient surgery were then put into a medium. As shown in FIG. 7A, the ovarian cancer cells grew normally before react with the cell population of the dendritic killer cells. And then, a step S202 of making the cell population of the dendritic killer cells co-cultivate with the target sample is performed. The abovementioned cell population of the dendritic killer cells will show its cytotoxicity by killing the tumor cells, a lot of the tumor cells then decreases as shown in FIG. 7B. Furthermore, the cell population of the dendritic killer cells will swallow the fragments of the tumor cells to present tumor-specific antigens of the above target sample. That is, the above cell population of the dendritic killer cells will become the tumor-specific cell population of dendritic killer cells. Finally, the tumor-specific cell population of dendritic killer cells is harvested in step S203 as the formulation disclosed in the present invention. Preferably, the cell population of dendritic killer cells presenting specific antigens used in the formulation has a concentration of 10⁶ cell/mL.

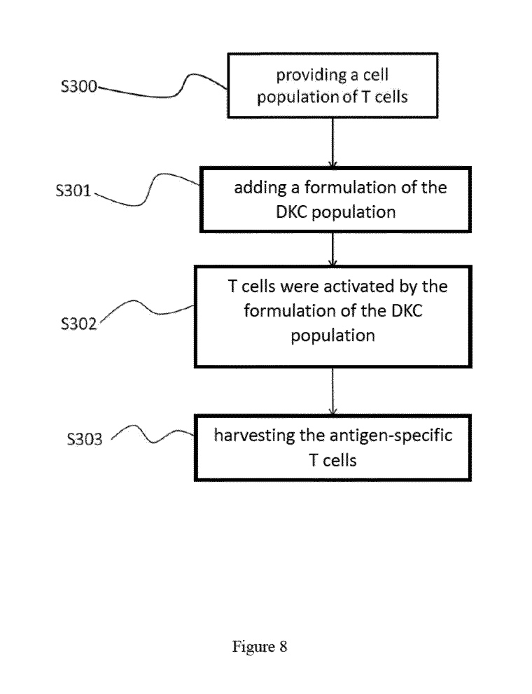

Please refer to FIG. 8; FIG. 8 is a flow chart showing a method according to an embodiment of the present invention for preparing specific T cells. The method comprises the following steps. As shown in step S300, a cell population of T cells is provided at first. And then, a formulation of preparing specific T cells is added to mix with the cell population of T cells in step S301. The cell population of the dendritic killer cells used in the formulation will present its tumor-specific antigens to activate T cells in step S302. In the step S302, the activated T cells identify the tumor-specific antigens so that those T cells become the tumor-specific T cells. Finally, the specific T cells are harvested in Step S303. Preferably, the formulation has a concentration of 10⁶ cell/mL, but the present invention is not limited thereto.

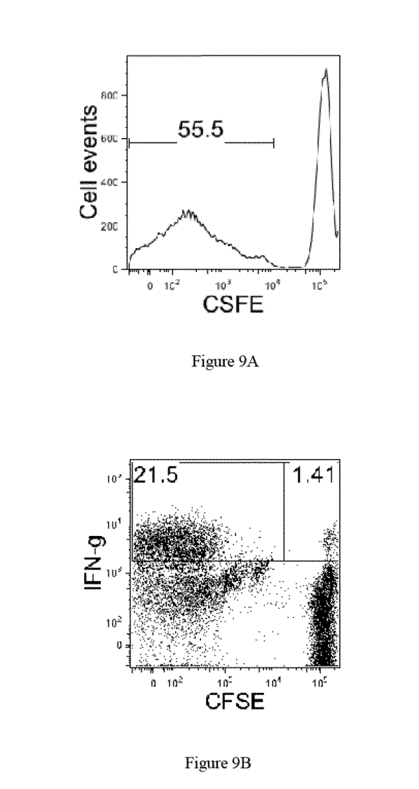

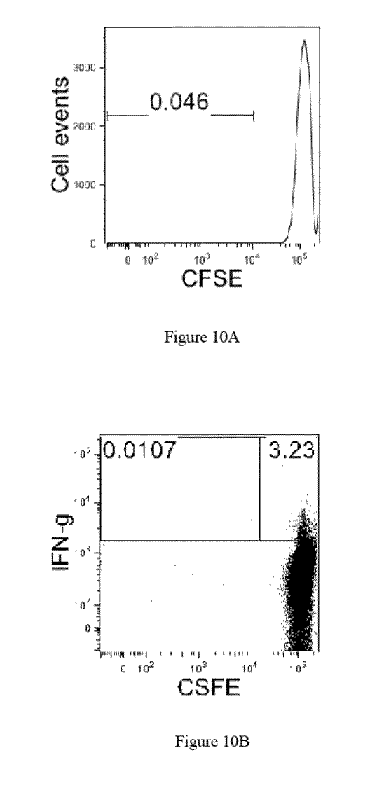

Please refer to FIG. 9A˜FIG. 10B. FIG. 9A and FIG. 9B are diagrams showing the result of activating T cells by the cell population of the dendritic killer cells presenting specific antigens according to an embodiment of the present invention; FIG. 10A and FIG. 10B are diagrams showing the result of activating T cells by the cell population of the dendritic killer cells without reacting with the tumor cells. As the abovementioned, the cell population of the dendritic killer cells presenting specific antigens can activate the T cells. Therefore, CD8⁺ T cells are sorted from the peripheral blood of the cancer patient, who is suffered from the ovarian cancer, and the sorted T cells are further marked by 6.5 μM of CFSE for observing the effect of activation of T cells. The abovementioned CFSE is a dye for quantifying the degree of cell proliferation. After each cell proliferation, the fluorescence intensity of CFSE will decrease. However, when CFSE labeling is performed optimally, approximately 7-8 cell divisions can be identified before the CFSE fluorescence is too low to be distinguished above the autofluorescence background. The present embodiment is detected by the flow cytometer, but it is not limited thereto. It is noted that the cell population of the dendritic killer cells, the cell population of the dendritic killer cells presenting specific antigens, the tumor cells and CD8⁺ T cells are both gathered from the same cancer patient.

As shown in FIG. 9A to FIG. 9B, FIG. 9A shows the result detected by the flow cytometer for observing the cleavage of the T cells after activating, and FIG. 9B shows the result of detecting IFN-γ existed in the T cells by the flow cytometer. That is, the present invention makes the cell population of the dendritic killer cells presenting specific antigens co-cultivate with the CD8⁺ T cells for 48 hours and further observes the cleavage of the T cells by the flow cytometer.

Please refer to FIG. 9A; the cell population of the dendritic killer cells presenting specific antigens can activate T cells to let 55% of them proliferate. On the other hand, the T cells activated by the cell population of the dendritic killer cells presenting specific antigens have IFN-γ therein as shown in the upper-left of FIG. 9B. That is to say, the activated T cells are specific for the antigens presented by the cell population of the dendritic killer cells so that they will produce IFN-γ and have cytotoxicity.

Please refer to FIG. 10A; it is clearly that only 0.046% of T cells are proliferated. That is, the cell population of dendritic killer cells, which is not reacted with the tumor cells, cannot activate the T cells. Furthermore, there is no IFN-γ detected within the T cells as shown in FIG. 10B. Therefore, the cell population of the dendritic killer cells, which is not reacted with the tumor cells, does not present specific antigens on its surface so that it cannot activate auto CD8⁺ T cells.

To sum up, DKC is a cell with function from both natural killer cell and dendritic cell. Although DKC plays an important role in immunoreactions, the content of the DKC in the human body is very rare. The trace DKC of the human blood can be expanded from 200-fold to 400-fold by the cultivating, screening and sorting technique disclosed in the present invention. Moreover, the DKC can kill the tumor cells by its ability of identifying tumor cells and further present the tumor-specific antigens to T cells; therefore, the tumor cells cannot avoid the identification of the immune cells. In the meantime, the target sample is gathered from the same cancer patient. That is, the cell population of the dendritic killer cells cultivated from a cancer patient will be reacted with the tumor cells obtained from the same cancer patient. After cultivating, the cell population of the dendritic killer cells presenting specific antigens will further activate CD8⁺ cells of the same cancer patient to form the tumor-specific T cells.

According to the abovementioned, it can be proved that the tumor cells would be recognized and killed by antigen-specific T cells, which was revealed by the method disclosed in the present invention. Furthermore, the efficiency of applying the cell population of the dendritic killer cells presenting specific antigens on preparing specific T cells has been further enhanced. Through contact a population of CD8⁺ T cells with the plurality of tumor antigen-pulsed human DKCs, we can obtain tumor antigen-specific CD8⁺ T cells. And administer the tumor antigen-specific CD8⁺ T-cells to the human subject to treat cancer by their own immune cells. That is, the specific T cells prepared ex vivo can be used as a novel method of immunization therapy. On the other hand, the method disclosed in the present invention is a platform for screening the specific antigens.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claim is:

1. A formulation for preparing specific T cells comprising at least a cell population of dendritic killer cells presenting specific antigens.

2. The formulation according to claim 1 further comprising a physiologically acceptable medium.

3. The formulation according to claim 1 further comprising a physiologically acceptable buffer.

4. The formulation according to claim 1, wherein a concentration of the cell population of dendritic killer cells presenting specific antigens is 10⁶ cells/mL.

5. The formulation according to claim 1, wherein the specific antigens are tumor-specific antigens.

6. A method for preparing a formulation of preparing specific T cells comprising the following steps:
providing a cell population of dendritic killer cells;
providing a target sample;
making the cell population of dendritic killer cells co-cultivate with the target sample; and
harvesting a cell population of dendritic killer cells presenting specific antigens after co-cultivating.

7. The method according to claim 6, wherein the cell population of the dendritic killer cells processes cytotoxicity against the target sample and presents a specific antigens of the target sample on its surface to form the cell population of dendritic killer cells presenting specific antigens.

8. The method according to claim 6, wherein the target sample is tumor cells from a cancer patient.

9. The method according to claim 8, wherein the specific antigens are tumor-specific antigens.

10. The method according to claim 8, wherein the target sample and the cell population of dendritic killer cells are both gathered from the cancer patient.

11. The method according to claim 6, wherein the cell population of dendritic killer cells is generated ex vivo by culturing dendritic killer cells from a human blood sample with a cytokine, and the cytokine comprises IL-15.

12. The method according to claim 6, wherein the method is a platform for screening specific antigens.

13. A method for preparing specific T cells comprising the following steps:
providing a cell population of T cells;
adding a formulation of preparing specific T cells and mix with the cell population of T cells; and
harvesting the specific T cells after cultivating.

14. The method according to claim 13, wherein the formulation comprises a cell population of dendritic killer cells presenting specific antigens.

15. The method according to claim 14, wherein the cell population of T cells is activated by the cell population of dendritic killer cells presenting specific antigens to form the specific T cells.

16. The method according to claim 13, wherein the specific antigens are tumor-specific antigens.

17. The method according to claim 16, wherein the specific T cells are tumor-specific T cells.

18. A composition containing a plurality of tumor antigen-pulsed human DKCs, wherein the plurality of tumor antigen-pulsed human DKCs is obtained by contacting a population of human DKCs with cells derived from a tumor, the population of human DKCs being HLA-G⁻ CD14⁻ CD19⁻ CD3⁻ CD56⁺ HLA-DR⁺.

19. A method of generating a plurality of tumor antigen-specific CD8⁺ T cells, the method comprising contacting a population of CD8⁺ T cells with the composition of claim 18, thereby generating a plurality of tumor antigen-specific CD8⁺ T cells.

20. A method of treating a tumor in a human subject in need thereof, the method comprising:
providing a plurality of tumor antigen-pulsed human DKCs that is prepared by contacting a population of human DKCs with cells derived from a tumor in the subject, the population of DKCs being HLA-G⁻ CD14⁻ CD19⁻ CD3⁻ CD56⁺ HLA-DR⁺;
contacting a population of CD8⁺ T-cells with the plurality of tumor antigen-pulsed human DKCs to generate tumor antigen-specific CD8⁺ T cells; and
delivering the tumor antigen-specific CD8⁺ T cells to the human subject.
